SEQUENCE LISTING (2 sequences)

SEQ ID NO: 1 - protein, 147 aa, Homo Sapiens
MGHFTEEDKATITSLWGKVNVEDAGGETLGRLLVVYPWTQRFFDSFGNLSSASAIMGNPKVKAHGKKVLTSLGDAIKHLDDLKGTFAQLSELHCDKLHVDPENFKLLGNVLVTVLAIHFGKEFTPEVQASWQKMVTAVASALSSRYH

SEQ ID NO: 2 - protein, 147 aa, Artificial Sequence
  Synthetic polypeptide
MGHFTEEDKATITSLWDKVNVEDAGGETLGRLLVVYPWTQRFFDSFGNLSSASAIMGNPKVKAHGKKVLTSLGDAIKHLDDLKGTFAQLSELHCDKLHVDPENFKLLGNVLVTVLAIHFGKEFTPEVQASWQKMVTAVASALSSRYH

(19) United States
(12) Patent Application Publication
(10) Pub. No.: US 2021/0380946 A1
(43) Pub. Date: Dec. 9, 2021

(54) REDUCED INTENSITY CONDITIONING WITH MELPHALAN

(71) Applicant: CHILDREN'S HOSPITAL MEDICAL CENTER, Cincinnati, OH (US)

(72) Inventor: Punam Malik, Cincinnati, OH (US)

(21) Appl. No.: 17/289,135

(22) PCT Filed: Nov. 1, 2018
(86) PCT No.: PCT/US2018/058790

§ 371 Date: Apr. 27, 2021

(51) Int. Cl. C12N 5/0789; A61K 31/198; A61K 35/28; C12N 15/86; A61P 7/00

(52) U.S. Cl. CPC C12N 5/0647; A61K 31/198; C12N 2740/15043; C12N 15/86; A61P 7/00; A61K 35/28

(57) ABSTRACT

A method of conditioning a subject for hematopoietic cell transplantation, wherein the method involves the use of a nitrogen mustard alkylating agent such as melphalan in an amount to achieve reduced-intensity conditioning.

### BACKGROUND OF THE INVENTION

Hematopoietic stem cell (HSC) transplantation is an essential course of treatment for a variety of indications and in such instances, the recipient subject is often treated with a myeloablative conditioning regime to destroy host HSCs. Myeloablative conditioning eliminates the initial competition from host cells, which the newly introduced transplanted cells may encounter.

While myeloblative conditioning is deemed important to achieve effective HSC transplantation, these regimes leave the recipient depleted of immune cells, and thus at a greater risk of infection and associated complication. Further, many of the substances used in the myeloblative conditioning regimens can cause damage to organs. Hence, better therapeutic options are needed for conditioning subjects for hematopoietic cell transplantation.

### SUMMARY OF THE INVENTION

The present disclosure is based, at least in part, on the unexpected discovery that patients having sickle cell disease who received melphalan for reduced-intensity conditioning followed by gene transfer therapy via transplantation of genetically engineered hematopoietic stem cells (HSCs) showed sustained stable genetically modified cells in blood that express γ-globin and lack of acute sickle event in at least 6 months after infusion. The results indicate that use of agents such as melphalan to achieve reduced-intensity conditioning for patients who need transplantation of hematopoietic cells could result in excellent safety, feasibility, minimal post-transplant toxicity, and a rapid count recovery.

Accordingly, the present disclosure features a method of conditioning a subject for hematopoietic cell transplantation, the method comprising (a) administering to a subject in need of the treatment a nitrogen mustard alkylating agent such as melphalan in an amount that leads to reduced-intensity conditioning in the subject, and optionally (b) transplanting a population of hematopoietic cells (e.g., hematopoietic stem cells) into the subject. The nitrogen mustard alkylating agent may be used in the method described herein in an amount that is lower than that for achieving myeloablative conditioning, for example, about 50-80% of the amount of the same agent for myeloablative conditioning.

In some embodiments, the nitrogen mustard alkylating agent is melphalan. The amount of melphalan used in any of the methods described herein may be about 120 mg/m² to about 160 mg/m². In specific examples, the amount of melphalan used in the method described herein is about 140 mg/m².

In some embodiments, the hematopoietic cells such as HSCs are genetically engineered. For example, the genetically engineered hematopoietic cells may comprise a viral vector carrying a gene of interest. In some examples, the viral vector is retroviral vector (e.g., a lentiviral vector, a foamy virus vector, or a y retroviral vector), an adenoviral vector, an adeno-associated viral vector, or a hybrid vector. The gene of interest may encode a γ-globin protein, which may be a human γ-globin protein. In some instances, the human γ-globin is a wild-type human γ-globin protein. Alternatively, the human γ-globin can be a mutated human γ-globin protein, which may have an enhanced binding affinity to the α-globin subunit. For example, the mutated human γ-globin protein may comprise a substitution at a position corresponding to position 17 of a wild-type human γ-globin protein (SEQ ID NO:1).

In any of the methods disclosed herein, the subject may be a human subject. In some embodiments, the subject may have, be suspected of having, or be at risk of a hemoglobinopathy or anemia. For example, the subject may be a human patient having thalassemia (e.g., β-thalassemia) or sickle cell anemia.

Also within the scope of the present disclosure are pharmaceutical compositions comprising one or more nitrogen mustard alkylating agents as disclosed herein (e.g., melphalan) for use to achieve reduced-intensity conditioning in a subject who is in need of hematopoietic cell transplantation, and uses of the nitrogen mustard alkylating agent for manufacturing a medicament for use in inducing reduced-intensity conditioning in a subject who is in need of the hematopoietic cell transplantation.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a diagram showing an exemplary lentiviral vector encoding a modified human γ-globin protein (SEQ ID NO:2).

FIG. 2 is a diagram illustrating the clinical study protocol described in Example 1.

FIG. 3 is a chart showing the express levels of fetal and sickle globin proteins in patients subject to the treatment disclosed herein.

### DETAILED DESCRIPTION OF THE INVENTION

HC transplantation is a known therapy for a range of indications, including those associated with a deficiency or other abnormality in a subject's hematopoietic system, genetic defects, etc. However, simply transplanting HCs is often insufficient to effectuate long-term therapeutic results, for example, complications arising from the interaction with endogenous cells in the subjects system often occur (e.g., competition between endogenous HSCs and the transplanted HSCs, leading to low level of engraftment or complete elimination of the transplanted HSCs).

To solve this problem, conventional hematopoietic cell transplantation typically involves myeloablative conditioning, which destroys host hematopoietic cells such as HSCs, thus allowing noncompetitive repopulation of gene-corrected donor HSCs. Generally, this is accomplished through a regime involving the use of maximally tolerated doses of one or more chemotherapeutics, either alone or in combination with radiation. Such a course of action however, often has various adverse side-effects. Many chemotherapeutics are harmful and deleterious to the subject's organs, and radiation can lead to a multitude of systemic problems.

On the other end, myeloablative conditioning was viewed as an important step to achieve high levels of transgene-modified HSC engraftment and transgene expression via providing adequate immunosuppression to prevent rejection of the transplanted hematopoietic cells. However, various side effects associated with myeloablative conditioning has significantly limited the application of HSC-mediated gene transfer therapy.

The present disclosure provides an improvement of the traditional myeloablative conditioning in association with HSC-mediated gene transfer therapy. Unexpectedly, it was observed in human patients that reduced-intensity conditioning by a nitrogen mustard alkylating agent followed by transplantation of genetically engineered HSCs adapted to express a transgene of human γ-globin showed successful engraftment of the engineered HSCs and expression of the transgene with minimal transplant toxicity and a rapid count recovery. The procedure showed significant efficacy in treating patients having sickle cell disease as an example.

Accordingly, provided here is an advantageous conditioning regimen (reduced-intensity conditioning regimen) for patients who need HSC-mediated gene transfer therapy to enhance the efficiency of HSC engraftment and transgene expression and reduce side effects commonly associated with myeloablative. Also provided herein are HSC-mediated gene transfer methods for treating a target disorder, in which a patient is subject to the reduced-intensity condition regimen as disclosed herein.

### I. Reduced-Intensity Conditioning Regimen

The reduced-intensity conditioning regimen disclosed herein involves administering to a subject (e.g., a human patient) who needs HSC transplantation an amount of a nitrogen mustard alkylating agent that is sufficient to result in reduced-intensity conditioning in the subject. This regimen would put a subject in a good condition for receiving HSC transplantation—to achieve some level of immune suppression such that the transplanted HSCs would not be rejected by the host immune system and to reduce side effects associated with myeloablative conditioning regimens commonly used in association with HSC transplantation, particularly HSC transplantation-mediated gene transfer therapy.

As used herein the term “condition” or “conditioning” in the context of a subject pretreatment in need of HC transplantation typically means destroying the bone marrow and immune system of the subject by a suitable procedure, partially or completely. “Myeloablative conditioning” means to destroy bone marrow cells substantially to ablate marrow hematopoiesis and not allow autologous hematologic recovery. “Reduced-intensity conditioning” means to destroy bone marrow cells to some extent such that marrow hematopoiesis is not completely ablated. In some instances, “reduced-intensity conditioning” can be achieved by using less chemotherapy and/or radiation than the standard myeloablative conditioning regimens, for example 50-80% (e.g., 55-75% or 60-70%) of the amount of a chemotherapeutic commonly used for myeloablative conditioning. Additional information of myeloablative conditioning and reduced-intensity conditioning can be found, e.g., in Gyurkocza et al. Blood, 124(3):344-353, 2014, the relevant disclosures of which are incorporated by reference for the purposes or subject matter referenced herein.

To perform the reduced-intensity conditioning regimen disclosed herein, a suitable amount of a nitrogen mustard alkylating agent, such as melphalan, can be administered to a subject in need of the treatment via a suitable route. Nitrogen mustard alkylating agents, derived from mustard gas, are a group of compounds capable of alkylating DNA and form inter-strand cross-links in DNAs. Such compounds are commonly used in cancer therapy. Nitrogen mustard alkylating agents typically contain the core structure of

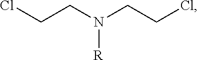

in which R is optionally substituted carbocyclyl, optionally substituted heterocyclyl, optionally substituted aryl, or optionally substituted heteroaryl. In some instances, R is optionally substituted carbocyclyl, optionally substituted aryl (e.g., substituted phenyl), or optionally substituted heteroaryl. Pharmaceutically acceptable salts, solvates, hydrates, polymorphs, co-crystals, tautomers, stereoisomers, and isotopically labeled derivatives are also within the scope of the present disclosure.

Examples of nitrogen mustard alkylating agents include, but are not limited to, mustine, cyclophosphamide, chlorambucil, uramustine, ifosfamid, melphalan, and bendamustine. In some embodiments, the nitrogen mustard alkylating agent for use in the methods disclosed herein is a melphalan compound. Melphalan, also known as sarcolysin, is a chemotherapy drug. The chemical structure of melphalan is shown below.

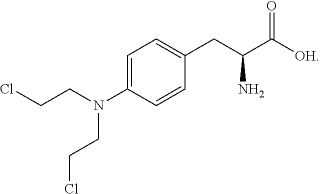 [SMILES: N[C@@H](Cc1ccc(N(CCCl)CCCl)cc1)C(=O)O]

A melphalan compound refers to melphalan, a pharmaceutically acceptable salt or ester thereof, or a derivative thereof. A derivative maintains the core structure noted above and similar alkylating activity, and may include one or more suitable substituents at positions where applicable and where valency permits.

Any of the nitrogen mustard alkylating agents disclosed herein (e.g., a melphalan compound such as melphalan) may be mixed with one or more pharmaceutically acceptable carriers, diluents, and/or excipienst to form a pharmaceutical composition for administration by a suitable route. A carrier, diluent, or excipient that is “pharmaceutically acceptable” includes one that is sterile and pyrogen free. Suitable pharmaceutical carriers, diluents, and excipients are well known in the art. The carrier(s) must be “acceptable” in the sense of being compatible with the inhibitor and not deleterious to the recipients thereof. See, e.g., Remington: The Science and Practice of Pharmacy 20th Ed. (2000) Lippincott Williams and Wilkins, Ed. K. E. Hoover.

A pharmaceutical composition comprising any of the nitrogen mustard alkylating agent such as a melphalan compound as described herein may be administered by any administration route known in the art, such as parenteral administration, oral administration, buccal administration, sublingual administration, or inhalation, in the form of a pharmaceutical formulation comprising the active ingredient, optionally in the form of a non-toxic organic, or inorganic, acid, or base, addition salt, in a pharmaceutically acceptable dosage form. In some embodiments, the administration route is oral administration and the formulation is formulated for oral administration.

In some embodiments, the pharmaceutical compositions or formulations are for parenteral administration, such as intravenous, intra-arterial, intra-muscular, subcutaneous, or intraperitoneal administration.

Formulations of the nitrogen mustard alkylating agent suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well-known to those skilled in the art.

In some embodiments, the pharmaceutical composition or formulation containing a nitrogen mustard alkylating agent may be suitable for oral, buccal or sublingual administration. Such pharmaceutical compositions may be in the form of tablets, capsules, ovules, elixirs, solutions or suspensions, which may contain flavoring or coloring agents, for immediate-, delayed- or controlled-release applications.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavoring agents, coloring matter or dyes, with emulsifying and/or suspending agents and with diluents such as water, ethanol, propylene glycol and glycerin, and combinations thereof.

In some embodiments, the pharmaceutical composition or formulation is suitable for intranasal administration or inhalation, such as delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoro-ethane, a hydrofluoroalkane, carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant. Capsules and cartridges (made, for example, from gelatin) for use in an inhaler or insufflator may be formulated to contain a powder mix of the nitrogen mustard alkylating agent and a suitable powder base such as lactose or starch.

The formulations may be presented in unit-dose or multi-dose containers, for example sealed ampoules or vials, and may be stored in a freeze-dried (lyophilized) condition requiring only the addition of the sterile liquid carrier immediately prior to use.

In some embodiments, the formulations can be pre-loaded in a unit-dose injection device, e.g., a syringe, for intravenous injection.

To perform the reduced-intensity conditioning regimen disclosed herein, an effective amount of the nitrogen mustard can be administered to a subject in need of the treatment via a suitable route (e.g., those described herein). An “effective amount,” “effective dose,” or an “amount effective to”, as used herein, refers to an amount of a nitrogen mustard alkylating agent as described herein that is effective in achieving the reduced-intensity conditioning in a subject who needs HSC transplantation therapy. Effective amounts vary, as recognized by those skilled in the art, depending on route of administration, excipient usage, and co-usage with other active agents.

In some instances, the amount of a nitrogen mustard alkylating agent for use in the reduced-intensity conditioning regimen disclosed herein is about 50-80% (e.g., about 55-75%, about 60-70%) of the effective amount of the same agent used for myeloablative conditioning as known in the art. For example, when melphalan is used for the reduced-intensity conditioning regimen, the amount of melphalan can range from 120-160 mg/m² (as opposed to the common dosage of 210 mg/m² for myeloablative conditioning. In one particular example, the amount of melphalan is about 140 mg/m². A physician in any event may determine the actual dosage which will be most suitable for any subject, which will vary with the age, weight and the particular disease or disorder to be treated or prevented.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The nitrogen mustard alkylating agent may be given to a subject by a single dose. If necessary, multiple doses may be given to the subject following routine practice. For example, a subject in need of an HC transplantation may be given a nitrogen mustard alkylating agent daily, every 2 days, every 3 days, or longer, prior to receiving the HC transplantation.

### II. Hematopoietic Cell Transplantation.

After or currently with reduced-intensity conditioning, HC such as HSC transplantation may be administered to the subject via a routine procedure (e.g., infusion). hematopoietic cells (HCs) refer to any cells having hematopoietic origin, include those lodged within the bone marrow (e.g., HSCs), cells differentiated therefrom (for example, those circulating in the blood such as red blood cells, white blood cells, and platelets), HCs such as HSCs derived from in vitro differentiation of stem cells (e.g., induced pluripotent stem cells or iPSCs).

Hematopoietic stem cell transplantation (HSCT) is the transplantation of multipotent hematopoietic stem cells, which may be derived from bone marrow, peripheral blood, umbilical cord blood, or from iPSCs. HCs can be obtained using conventional methods. For example, HCs can be isolated from from bone marrow, peripheral blood cells, and/or umbilical cord blood. One or more mobilizing agents, such as Plexifor, may be used to increase the availability of HCs. Alternatively, the HCs can be derived from stem cells (e.g., induced pluripotent stem cells which can be differentiated from somatic cells such as skin cells). The HCs can be cultured ex vivo prior to transplantation to a subject.

In some embodiments, the HCs may be isolated from the same subject (autologous), cultured ex vivo when needed, and be transplanted back to the subject.

Administration of autologous cells to a subject may result in reduced rejection of the stem cells as compared to administration of non-autologous cells. Alternatively, the HCs can be allogenic, i.e., obtained from a different subject of the same species. For allogeneic HC transplantation, allogeneic HCs may have a HLA type that matches with the recipient.

In any of the HC transplantation therapies described herein, suitable HCs such as

HSCs can be collected from the ex vivo culturing method described herein and mixed with a pharmaceutically acceptable carrier to form a pharmaceutical composition, which is also within the scope of the present disclosure.

In some instances, when applicable the transplanted cells may be modified to deliver a therapeutic effect. For example, but in no way defining or limiting, such cells may be genetically engineered cells to contain a gene to encode for a protein which the subject was previously deficient because of a mutation in his/her own genetic makeup. In other instances, the cells may contain a gene which is modified to express for increased amounts of a protein to counteract or offset another protein or product in the subject. In some instances, this may be accomplished by transducing the cells with a viral vector. A “vector”, as used herein is any vehicle capable of facilitating the transfer of genetic material (e.g., a shRNA, siRNA, ribozyme, antisense oligonucleotide, protein, peptide, or antibody) to a cell in the subject, such as HCs. In general, vectors include, but are not limited to, plasmids, phagemids, viruses, and other vehicles derived from viral or bacterial sources that have been manipulated by the insertion or incorporation of a sequence encoding a gene of interest. Viral vectors include, but are not limited to nucleic acid sequences from the following viruses: retrovirus; lentivirus; adenovirus; adeno-associated virus; SV40-type viruses; polyoma viruses; Epstein-Barr viruses; papilloma viruses; herpes virus; vaccinia virus; polio virus. One can readily employ other vectors not named but known to the art.

Viral vectors may be based on non-cytopathic eukaryotic viruses in which nonessential genes have been replaced with a sequence encoding a gene of interest. Non-cytopathic viruses include retroviruses (e.g., lentivirus, gamma-retrovirus, or foamy virus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are known in the art.

Other viral vectors include adeno-viruses and adeno-associated viruses, which are double-stranded DNA viruses that have also been approved for human use in gene therapy. The adeno-associated virus can be engineered to be replication deficient and is capable of infecting a wide range of cell types and species.

Other vectors include plasmid vectors. Plasmid vectors have been extensively described in the art and are well known to those of skill in the art. See, e.g., Sambrook et al. Molecular Cloning: A Laboratory Manual. Cold Spring Harbor Laboratory Press; 4th edition (Jun. 15, 2012). Exemplary plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA, such as a sequence encoding a γ-globin gene.

It is well known in the art viral vectors can encode for gene of interest, which can be then delivered via the vector to the cells to be transplanted. These genes of interest can be exploited to supply a therapeutic protein or correct for another abnormality or deficiency. It is known in the art, this can be accomplished by selecting a gene of interest which encodes for the appropriate property, and it is well known that both wild-type and mutated genes can be used. In the present case, a lentivirus vector was modified to carry a human γ-globin gene which was mutated at a position corresponding to position 17 of the wild-type γ-globin gene. The mutated human γ-globin gene is used to genetically correct sickle cell anemia or thalassemia or reduce symptoms thereof. This was carried out by performing the method comprising the steps of identifying a subject in need of treatment for sickle cell anemia or thalassemia; transfecting autologous HCs with a modified lentivirus comprising the mutated human γ-globin gene; and transplanting the transfected HCs into the subject.

In some examples, the HSCs described herein (e.g., human adult HSCs) can be genetically engineered to express a gene of interest suitable for treatment of a target disease, for example, a γ-globin for use in treating anemia, such as sickle cell anemia and thalassemia. See, e.g., US20110294114 and WO2015/117027, the relevant teachings of each of which are incorporated by reference for the purposes or subject matter referenced herein.

Any of the HC cells disclosed herein may be administered to a subject who has undergone or is undergoing the reduced-intensity conditioning regimen as disclosed herein via a suitable route, for example, intravenous infusion. In some embodiments, the subject may be given at least 10⁵ cells per infusion, for example, at least 10⁶, at least 10⁷, or at least 10⁸ cells. Typically, HC transplantation would be carried out after the reduced-intensity conditioning so as to give time for the host HCs to be inhibited or eliminated by the nitrogen mustard alkylating agent. The HC cells may be given to a subject 12 hours after the reduced-intensity conditioning, 24 hours after the reduced-intensity conditioning, 36 hours after the reduced-intensity conditioning, 48 hours after the reduced-intensity conditioning, 72 hours after the reduced-intensity conditioning, one week after the reduced-intensity conditioning, or longer.

In some embodiments, the HC transplantation can be co-used with a therapeutic agent for a target disease, such as those described herein. The efficacy of the stem cell therapy described herein may be assessed by any method known in the art and would be evident to a skilled medical professional. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject.

### Therapeutic Applications

The methods disclosed herein, involving any of the reduced-intensity conditioning regimens disclosed herein followed by hematopoietic cell transplantation also disclosed herein can be used for treating suitable target diseases, particularly those that require gene transfer therapy.

The term “treating” as used herein refers to the application or administration of a composition including one or more active agents to a subject, who has a target disease, a symptom of the target disease, or a predisposition toward the target disease, with the purpose to cure, heal, alleviate, relieve, alter, remedy, ameliorate, improve, or affect the disease, the symptoms of the disease, or the predisposition toward the disease.

The subject to be treated by the methods described herein can be a human (e.g., a male or a female of any age group). In some instances, the subject can be a pediatric subject (e.g., an infant, child, or an adolescent) or an adult subject (e.g., a young adult, a middle-aged adult, or a senior adult). The subject may also include any non-human animals including, but not limited to a nonhuman mammal such as cynomolgus monkey or a rhesus monkey. In certain embodiments, the non-human animal is a mammal, a primate, a rodent, an avian, an equine, an ovine, a bovine, a caprine, a feline, or a canine. The non-human animal may be a male or a female at any stage of development. The non-human animal may be a transgenic animal or a genetically engineered animal.

In some embodiments, the subject (e.g., a human subject), may have, be suspected of having, be at risk of having, or be predisposed to having a disease that can be treated by gene transfer therapy, for example, a genetic disorder. In some instances, the subject is a human patient having a hemoglobinopathy, which refers to a disorder associated with a genetic defect that results in abnormal structure of one of the globin polypeptide of hemoglobin or reduction of the globin polypeptide, e.g., alpha- (α-), beta- (β-), or gamma- (γ-) globin. Common hemoglobinopathies include sickle-cell disease and thalassemia such as β-thalassemia. In some instances, the subject is a human patient having anemia, such as sickle-cell anemia, congenital dyserythropoietic anemia, and thalassemia such as β-thalassemia.

In specific examples, the methods described herein aim at treating sickle cell disease (SCD). SCD affects the β-globin gene and is one of the most common genetic defects, resulting in the production of a defective sickle-globin (HbS, comprised of two normal α-globin and two β/sickle-globin molecules). HbS polymerizes upon deoxygenation and changes the shape of discoid red blood cells (RBCs) to bizarre sickle/hook shapes. Sickled RBCs clog the microvasculature, causing painful acute organ ischemic events and chronic organ damage that foreshortens the life span of SCD patients to 45 years. This disease affects over 110,000 Americans, with 1000 newborns with SCD born every year and nearly 1000 babies born with this disease annually in Africa.

Fetal hemoglobin (HbF, comprised of α and γ globins, α₂γ₂) is produced during the fetal life and the first 6-9 months of age and has strong anti-sickling properties and protects the infant from sickling in the first year of life. Indeed, individuals with hereditary persistence of HbF that have SCD are asymptomatic. Hydroxyurea, a chemotherapeutic drug that increases HbF, is FDA-approved for ameliorating symptoms of SCD. However, hydroxyurea does not work for all patients, and due to daily life-long intake, is associated with poor compliance. Hence, better therapeutic options are needed for SCD.

In some embodiments, the HSCs used in the methods described herein are genetically modified to express a γ-globin, which can form HbF in a recipient of the HSCs, who can subject to the reduced-intensity conditioning before the transplant.

The γ-globin protein may be of any suitable species, for example, human, monkey, chimpanzee, pig, mouse, rat, etc. In some instances, the γ-globin protein may be a wild-type protein. In others, the γ-globin protein may be a mutated form of a wild-type γ-globin protein, which retains substantially similar bioactivity as the wild-type counterpart and may have an increased binding affinity to the α-globin subunit, thereby forming fetal hemoglobin (α₂γ₂) at a high level so as to compete against the defective adult hemoglobin (α₂β₂, in which the β-chain is defective). Such a γ-globin mutant may comprise a substitution at position 17 of a wild-type counterpart (e.g., a G→D substitution). In some instances, the γ-globin mutant contains a substitution at position 17 of a wild-type counterpart and share a sequence homology of at least 85% (e.g., at least 90%, at least 95%, at least 97%, at least 98% or above) relative to the wild-type counterpart.

A functional mutant of a wild-type γ-globin would maintain substantially similar bioactivity of the native counterpart and share a high amino acid sequence homology with the native counterpart (e.g., at least 85%, at least 90%, at least 95%, at least 97%, at least 98% or above). The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some instances, a functional variant may contain conservative amino acid residue substitutions relative to the native counterpart. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

An exemplary wild-type human γ-globin protein and a mutant form thereof are provided below:
Amino acid sequence of a wild-type human γ-globin protein:

(SEQ ID NO: 1)
MGHFTEEDKATITSLWGKVNVEDAGGETLGRLLVVYPWTQRFFDSFGNL
SSASAIMGNPKVKAHGKKVLTSLGDAIKHLDDLKGTFAQLSELHCDKLH
VDPENFKLLGNVLVTVLAIHFGKEFTPEVQASWQKMVTAVASALSSRYH

Amino acid sequence of a mutant human γ-globin protein (substitution in boldface and underlined):

(SEQ ID NO: 2)
MGHFTEEDKATITSLWDKVNVEDAGGETLGRLLVVYPWTQRFFDSFGNL
SSASAIMGNPKVKAHGKKVLTSLGDAIKHLDDLKGTFAQLSELHCDKLH
VDPENFKLLGNVLVTVLAIHFGKEFTPEVQASWQKMVTAVASALSSRYH

Other exemplary γ-globin proteins are well known in the art and can be retrieved from publically available gene database such as GenBank, using the above-noted sequences as queries. Examples include GenBank Accession nos. P02099.2, NP_001164974.1, and NP_001040611.2

Where it is desirable, the subject can further receive a second HC transplantation after the transplantation of the first population of HCs. The second HC transplantation can be performed any time after the first HC transplantation. For example, the second HC transplantation can be performed about 3 days or longer, including 4 days, 5 days, 6 days, 7 days, 2 weeks, 3 weeks, 4 weeks, or longer, after the first HC transplantation.

To perform the method described herein, an effective amount of a nitrogen mustard alkylating agent can be administered to a human subject having in need of an HC transplantation via a suitable route to achieve a reduced-intensity condition. One or more populations of HCs, modified or wild-type, may then be transplanted into the subject. The nitrogen mustard alkylating agent could induce apoptosis of the endogenous HCs and enhance engraftment of the donor HCs, thereby effective in treating SCA.

### Kits for Use in Conditioning Subjects for HC Transplantation

The present disclosure also provides kits for use in conditioning a subject in need of the treatment (e.g., a subject with a genetic disorder such as hemoglobinopathy) for HC transplantation. Such kits can include one or more containers comprising a nitrogen mustard alkylating agent, and optionally, one or populations of HC cells, which may be genetically engineered

In some embodiments, the kit can comprise instructions for use in accordance with any of the methods described herein. The included instructions can comprise a description of administration of the nitrogen mustard alkylating agent for conditioning a subject for HC transplantation as described herein. The kit may further comprise a description of selecting an individual suitable for treatment based on identifying whether that individual is, e.g., has or suspected of having hemoglobinopathy or other related diseases as described herein. In still other embodiments, the instructions comprise a description of administering the nitrogen mustard alkylating agent and/or the HCs to an individual in need of the treatment.

The instructions relating to the use of a nitrogen mustard alkylating agent generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The label or package insert indicates that the composition is used for conditioning subject for HC transplantation. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a mini-pump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a nitrogen mustard alkylating agent as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds.(1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLE 1

### Reduced Intensity Conditioning Followed by Hematopoietic Cell Transplantation for Treating Sickle Cell Anemia

A clinical trial study was designed and carried out to determine whether transfer of a fetal hemoglobin gene (γ-globin) using a lentivirus vector (gene transfer) into human blood making cells is safe and feasible in patients with sickle cell disease. For example, the safety of bone marrow collection, gene transfer and chemotherapy conditioning in subjects with SCD is to be evaluated and the feasibility of obtaining sufficient autologous gene modified stem cells that can engraft the subject with SCD is to be evaluated.

### Inclusion Criteria:

between 3 and 35 years of age;
have sickle cell disease (HbSS/HbS-β0/HbS-β+);
have severe sickle cell disease (defined as having three (3) or more vaso-occlusive crises requiring intravenous pain medication, two (2) or more acute chest syndrome events in the past two (2) years, or one (1) acute chest syndrome events requiring intensive care unit admission);
have actively refused hydroxyurea, do not have access to hydroxyurea, or hydroxyurea has failed to work;
are adults who do not have an HLA matched sibling or who do, but have actively refused an allogenic HC transplant; and
have adequate Functional Status to withstand HC transplant.

### Exclusion Criteria:

Subjects who have had stroke;
Children with HLA matched siblings;
Have received a prior HC transplant;
Have an active malignant disease;
Are sero-positive for HIV;
Are pregnant; or
Are or have been on and investigational agent in the last thirty (30) days.

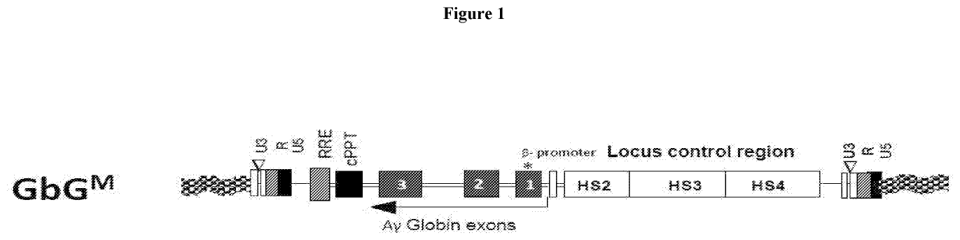

Bone marrow was the source of autologous HSC. Plerixafor mobilization-apheresis based stem cell collection was performed to harvest bone marrow cells multiple times from adult subjects. A modified γ-globin lentiviral vector is used to produce genetically modified HSCs. A diagram of this lentiviral vector is provided in FIG. 1, which encodes a mutant γ-globin protein disclosed above. Further information about the lentiviral vector and the mutant γ-globin protein can be found in US20150315611 and WO2015/117027, the relevant content of each of which is incorporated by reference for the purpose and subject matter referenced herein.

Two human patients were subject to this study:
Subject 1, 35 year old with baseline Hb of 8.5 g/dL; having 48 acute events from 24 month to 6 month before the treatment (3 events/month). Had multiple vaso-occlusive crises, acute chest, leg ulcer, chronic pain. Chronic opiate used.
Subject 2, 25 year old with baseline Hb of 8.5-9.5 g/dL; having 20 acute sickle events from 24 months to 6 months before the treatment. Experienced multiple VOC and ACS and chronic pain.

Hematopoietic stem cells were collected from the two subjects (from bone marrow or PMBC) and CD34⁺ cells were isolated. The lentiviral vector encoding the γ-globin protein was delivered into the enriched CD34⁺ cells to produce genetically engineered HSC cells adapted to express the γ-globin protein. Each of subjects was given 140 mg/m² melphalan by a single dose, followed by infusion or the genetically engineered HSC cells.

The subject information and treatment conditions are provide in Table 1 below:

TABLE 1
Subject Information and CD34 HSC Treatment Conditions

| | Age at infusion | CD34 Dose | CD34 Bulk VCN |
|---|---|---|---|
| Subject ID | (years) | (×10⁶/kg) | (copies/cell) |
| Subject 1 | 35 | 1.03 | 0.2 |
| Subject 2 | 25 | 6.9 | 0.5 |

Table 2 below shows neutropenia and thrombocytopenia post-melphalan treatment.

TABLE 2
Neutropenia and Thrombocytopenia Post-Melphalan

| | Days ANC >500 | |
|---|---|---|
| Subject ID | (Absolute Neutrophil Count) | Days Platelets <50 |
| Subject 1 | 9 | 13 |
| Subject 2 | 7 | 8 |

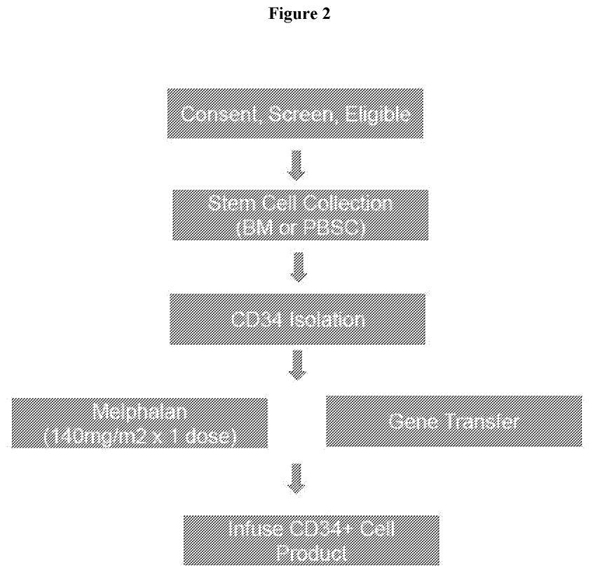

FIG. 2 provides an exemplary treatment regimen for reduced-intensity conditioning followed by HSC transplant for gene transfer therapy.

The subjects were followed for one (1) year and six (6) months, respectively. Both subjects experienced minimal adverse events, including chronic pain and chemotherapy related toxicity, including grade 2-3 mucositis, temporary cytopenia, and temporary mild evelvations in transaminases).

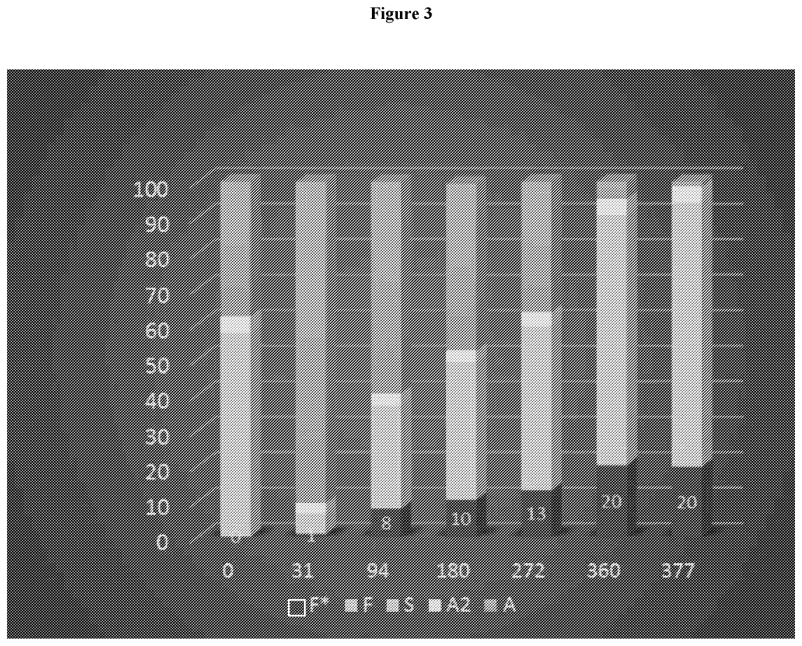

After one (1) year, as seen in FIG. 3 and below, subject 1 showed:
Stable gene marking in all lineages;
Immunoenzymatic staining assay shows it is highly polyclonal;
0 to +6 months: 3 acute sickle events requiring intravenous opiates and chronic pain despite HbS<30%; and
6 to 12 months: 1 sickle event requiring oral pain medicine.

| | Hb F* + F + A2 | HbS |
|---|---|---|
| % | 31 | 68 |
| g/dL | 3.4 | 7.2 |

After six (6) months, subject 2 has not had an acute sickle event.

Both subjects exhibit sustained stable genetically modified cells in blood and bone marrow and experienced minimal post-transplant toxicity with rapid count recovery.

In sum, early results from 2 SCA patients treated with a modified γ-globin delivered by a lentiviral vector and a reduced-intensity conditioning autologous HSC transplant showed excellent safety, feasibility, minimal post-transplant toxicity, and a rapid count recovery. One subject showed sustained genetically modified cells in blood and bone marrow one year following infusion and the second subject showed a similar trajectory.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method of conditioning a subject for hematopoietic cell (HC) transplantation, the method comprising:
(a) administering to a subject in need of a cell transplantation a nitrogen mustard alkylating agent in an amount leading to a reduced-intensity conditioning.

2. The method of claim 1, wherein the amount of the nitrogen mustard alkylating agent is about 50-80% of the amount of the same the nitrogen mustard alkylating agent that achieve myeloablative conditioning.

3. The method of claim 1, wherein the nitrogen mustard alkylating agent is melphalan.

4. The method of claim 3, wherein the amount of melphalan is about 120 mg/m² to about 160 mg/m².

5. The method of claim 4, wherein the amount of melphalan is about 140 mg/m².

6. The method of claim 1, further comprising:
(b) transplanting a population of hematopoietic cells into the subject.

7. The method of claim 6, wherein the hematopoietic cells are hematopoietic stem cells.

8. The method of claim 6, wherein the population of hematopoietic cells comprise genetically engineered hematopoietic cells.

9. The method of claim 8, wherein the genetically engineered hematopoietic cells are transfected with a viral vector which carries a gene of interest.

10. The method of claim 9, wherein the viral vector is a retroviral vector, an adenoviral vector, an adeno-associated viral vector, or a hybrid vector.

11. The method of claim 10, wherein the viral vector is a retroviral vector, which is a lentiviral vector, a foamy virus vector, or a gamma retroviral vector.

12. The method of claim 9, wherein the gene of interest encodes a gamma-globin protein.

13. The method of claim 12, wherein the gamma-globin protein is a human gamma-globin protein.

14. The method of claim 13, wherein the human gamma-globin is a wild-type human gamma-globin protein.

15. The method of claim 14, wherein the human gamma-globin is a mutated human gamma-globin protein, which comprises a substitution at a position corresponding to position 17 of a wild-type human gamma-globin protein.

16. The method of claim 1, wherein the subject is a human patient.

17. The method of claim 16, wherein the human patient has, is suspected of having, or is at risk for a hemoglobinopathy.

18. The method of claim 16, wherein the human patient has anemia.

19. The method of claim 18, wherein the anemia is thalassemia or sickle cell anemia.

20. The method of claim 19, wherein the thalassemia is β-thalassemia.
